(19) United States
(12) Patent Application Publication
(10) Pub. No.: US 2021/0000493 A1
(43) Pub. Date: Jan. 7, 2021

(54) TORQUE ACCESSORY FOR SUPPORT CATHETER

(71) Applicant: BOSTON SCIENTIFIC LIMITED, Hamilton (BM)

(72) Inventors: SHARATH BADADAMATH, HOSAPETE (IN); ADITYA DHANOTIYA, INDORE (IN)

(73) Assignee: BOSTON SCIENTIFIC LIMITED, HAMILTON (BM)

(21) Appl. No.: 16/917,169

(22) Filed: Jun. 30, 2020

Related U.S. Application Data
(60) Provisional application No. 62/869,531, filed on Jul. 1, 2019.

(51) Int. Cl. A61B 17/22

(52) U.S. Cl. CPC A61B 17/22; A61M 2025/09116; A61B 2017/22094; A61B 2017/22038

(57) ABSTRACT

A guidewire control assembly may hold a guidewire stationary while allowing for rotation of a catheter shaft disposed over the guidewire. The guidewire control assembly may include a housing with a passage for receiving the guidewire, a clutch mechanism connected to the housing and actuatable to selectively grip and release the guidewire disposed in the passage, and an interface rotatably connected to the housing. The interface may receive the catheter hub attached to the catheter shaft and may allow for torquing the catheter hub.

### CROSS-REFERENCE TO RELATED APPLICATIONS

This application claims the benefit of priority under 35 U.S.C. § 119 to U.S. Provisional Application Ser. No. 62/869,531, filed Jul. 1, 2019, the entirety of which is incorporated herein by reference.

### TECHNICAL FIELD

The disclosure pertains to medical devices and more particularly to devices for arterial or vascular intervention techniques, and methods for using such medical devices.

### BACKGROUND

A wide variety of medical devices have been developed for medical use including, for example, medical devices utilized to treat chronic total occlusion (CTO) and peripheral arterial disease (PAD). In these procedures a support catheter may be used to provide support and column strength to the guidewire. During the procedure, the support catheter hub is often torqued while holding it in the user's primary hand, while the user simultaneously clutches the guidewire with the little finger to avoid torquing the guidewire. The user torques the catheter shaft using the opposite hand with a pinch grip. Clutching the guidewire with the little finger is difficult due to the small diameter and hydrophilic nature of the guidewire, and with procedure times of 15-20 minutes, this often results in muscle strain. Holding the guidewire in this manner may result in kinking of the guidewire and/or catheter shaft. There is an ongoing need to provide alternative medical devices for these procedures as well as alternative methods for manufacturing and using the medical devices.

### SUMMARY

This disclosure provides design, material, manufacturing method, and use alternatives for medical devices.

In a first example, a guidewire control device comprises a housing having a passage for receiving a guidewire, a clutch connected to the housing and actuatable to selectively grip and release the guidewire disposed in the passage, and an interface rotatably connected to the housing, the interface configured to receive a catheter hub and torque the catheter hub.

In addition or alternatively, and in another example, the clutch incudes an engagement member configured to grip the guidewire and prevent rotation of the guidewire while the interface rotates.

In addition or alternatively, and in another example, the engagement member includes first and second opposing pads configured to be moved toward each other and into contact with the guidewire.

In addition or alternatively, and in another example, the clutch further includes first and second opposing grips attached to the first and second opposing pads, respectively, the first and second opposing grips configured to move between a release configuration and a gripping configuration, wherein when in the release configuration, the first and second opposing grips extend radially outward from an outer surface of the housing, and when in the gripping configuration, the first and second opposing grips are disposed radially inward toward the passage.

In addition or alternatively, and in another example, the first and second opposing grips are biased in the release configuration.

In addition or alternatively, and in another example, the interface includes a proximal end defining a lumen for receiving the guidewire, the proximal end having at least one protrusion configured to rotate within at least one channel on an inner surface of the passage of the housing.

In addition or alternatively, and in another example, the interface includes a distal end defining a cavity having internal threading configured to receive an externally threaded proximal end of the catheter hub.

In addition or alternatively, and in another example, the guidewire control device further comprises an accessory device including an actuator having a lumen configured to receive a catheter, the actuator having a distal end defining an actuatable clamp configured to engage the catheter, the accessory device further including a sleeve configured to actuate the actuatable clamp.

In addition or alternatively, and in another example, the actuatable clamp includes a plurality of deflectable arms moveable between a release configuration in which the catheter slides through the lumen, and a gripping configuration in which the plurality of deflectable arms engage the catheter and prevent the catheter from moving axially or rotationally.

In addition or alternatively, and in another example, the sleeve defines a proximal chamber configured to receive the plurality of deflectable arms in the release configuration, a central chamber configured to receive the plurality of deflectable arms in the gripping configuration, and a lumen configured to receive the catheter, wherein when the plurality of deflectable arms of the actuator are moved from the proximal chamber into the central chamber, the plurality of deflectable arms are compressed radially inward into engagement with the catheter.

In another example, a guidewire control assembly comprises a guidewire clamping device including a proximal housing having a passage for receiving a guidewire, a clutch connected to the proximal housing and actuatable to selectively grip and release the guidewire disposed in the passage, and a distal housing rotatably connected to the proximal housing, the distal housing configured to receive a catheter hub, a catheter clamping device including a first member having a lumen configured to receive a catheter and a distal end defining an actuatable clamp configured to engage the catheter, the catheter clamping device further including a second member having a cavity configured to receive and actuate the actuatable clamp.

In addition or alternatively, and in another example, the clutch incudes an engagement member configured to grip the guidewire and prevent rotation of the guidewire while the distal housing rotates, the engagement member including first and second opposing pads configured to be moved toward each other and into contact with the guidewire.

In addition or alternatively, and in another example, the clutch further includes first and second opposing grips attached to the first and second opposing pads, respectively, the first and second opposing grips configured to move between a release configuration and a gripping configuration, wherein when in the release configuration, the first and second opposing grips extend radially outward from an outer surface of the proximal housing, and when in the gripping configuration, the first and second opposing grips are disposed radially inward toward the passage.

In addition or alternatively, and in another example, the first and second opposing grips are biased in the release configuration.

In addition or alternatively, and in another example, the distal housing includes a proximal end defining a lumen for receiving the guidewire, the proximal end having at least one protrusion configured to rotate within at least one channel on an inner surface of the passage of the distal housing.

In addition or alternatively, and in another example, the distal housing includes a distal end defining a cavity having internal threading configured to receive an externally threaded proximal end of the catheter hub.

In addition or alternatively, and in another example, the actuatable clamp includes a plurality of deflectable arms moveable between a release configuration in which the catheter slides through the lumen, and a gripping configuration in which the plurality of deflectable arms engage the catheter and prevent the catheter from moving axially or rotationally.

In addition or alternatively, and in another example, the second member defines a lumen having a proximal region configured to receive the plurality of deflectable arms in the release configuration a middle region configured to receive the plurality of deflectable arms in the gripping configuration, and a distal region configured to receive the catheter, wherein when the plurality of deflectable arms of the actuatable clamp are moved from the proximal region into the middle region, the plurality of deflectable arms are compressed radially inward into engagement with the catheter.

In another example, a method of torquing a catheter disposed over a guidewire comprises loading a catheter shaft attached to a catheter hub over a guidewire, loading a guidewire control device onto the guidewire, the guidewire control device including a housing having a passage for receiving the guidewire, a clutch connected to the housing and actuatable between a release configuration in which the guidewire is freely moveable within the passage and a gripping configuration in which the guidewire is prevented from rotational and axial movement relative to the housing, and an interface rotatably connected to the housing, wherein the clutch is in the release configuration, attaching the catheter hub to the interface, actuating the clutch to the gripping configuration thereby preventing rotational and axial movement of the guidewire relative to the housing, and torquing the catheter by rotating the interface.

In addition or alternatively, and in another example, before loading the catheter shaft over the guidewire, the method further comprises loading an accessory device onto the catheter shaft, the accessory device including an actuator having a lumen configured to receive the catheter shaft, the actuator having a distal end defining an actuatable clamp having a release configuration in which the catheter shaft is freely moveable within the lumen and a gripping configuration in which the catheter shaft is prevented from rotational and axial movement relative to the accessory device, the accessory device further including a sleeve configured to actuate the actuatable clamp, wherein the actuatable clamp is in the release configuration, actuating the accessory device by moving the sleeve over the actuatable clamp thereby moving the actuatable clamp into the gripping configuration, and wherein torquing the catheter includes rotating the interface and the accessory device in the same direction.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective view of an exemplary guidewire control device connected to a catheter hub;

FIG. 2 is an exploded view of the guidewire control device and catheter hub of FIG. 1;

FIG. 3A is a cross-sectional view of the guidewire control device and catheter hub of FIG. 1, with the clutch mechanism in a release configuration;

FIG. 3B is a cross-sectional view of the guidewire control device and catheter hub of FIG. 1, with the clutch mechanism in a gripping configuration;

FIG. 4 shows the guidewire control device of FIG. 1 in a release configuration and being connected to a catheter hub;

FIG. 5 shows the guidewire control device of FIG. 1 in an gripping configuration;

FIG. 6 is a perspective view of an exemplary accessory device in a release configuration;

FIG. 7 is an exploded view of the accessory device of FIG. 6;

FIG. 8 is a cross-sectional view of the accessory device of FIG. 6;

FIG. 9 is a cross-sectional view of the accessory device of FIG. 6 in a gripping configuration;

FIG. 10 illustrates an assembly of the guidewire control device of FIG. 1 and the accessory device of FIG. 6 on a catheter, each device in a release configuration; and

FIG. 11 illustrates the assembly of FIG. 10 with each device in a gripping configuration.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “an example”, “some embodiments”, “some examples”, “other embodiments”, “other examples” etc., indicate that the embodiment(s) or example(s) described may include a particular feature, structure, or characteristic, but every embodiment or example may not necessarily include the particular feature, structure, or characteristic. Moreover, such phrases are not necessarily referring to the same embodiment or example. Further, when a particular feature, structure, or characteristic is described in connection with one embodiment or example, it would be within the knowledge of one skilled in the art to effect the particular feature, structure, or characteristic in connection with other embodiments or examples, whether or not explicitly described, unless clearly stated to the contrary. That is, the various individual elements described below, even if not explicitly shown in a particular combination, are nevertheless contemplated as being combinable or arrangeable with each other to form other additional embodiments or examples or to complement and/or enrich the described embodiment(s) or example(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Treating patients with diseased arteries often involves crossing an occlusion with a guidewire. A support catheter may be used to provide column support to the guidewire. During the procedure, the support catheter hub is often torqued while holding it in the user's primary hand while simultaneously clutching the guidewire with the little finger of the primary hand to avoid torquing the guidewire. The user may then torque the catheter using a pinch grip with the second hand. This procedure has disadvantages including the difficulties of clutching a small diameter guidewire with the little finger, especially as the guidewire often has a hydrophilic coating and is usually wet. The procedure often lasts 15-20 minutes, and clutching the guidewire and/or torquing the catheter multiple times during the procedure using the hands often results in muscle strain. The catheter shaft and hub generally have different diameters and torquing both simultaneously may transfer differential torque due to differences in diameters, and this may result in kinking and damaging the catheter shaft. Devices for simultaneously gripping the guidewire to hold it stationary while torquing the catheter shaft are needed.

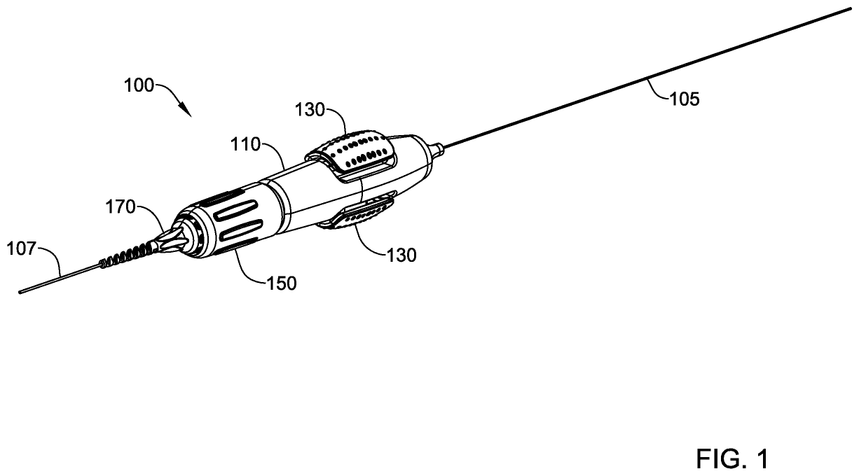

As will be described in greater detail below, FIG. 1 illustrates an example guidewire control device 100 including a housing 110, an attached clutch mechanism 130, and an interface 150 rotatably coupled to the housing 110. The housing 110 is configured to receive a guidewire 105, and the interface 150 is configured to receive a catheter hub 170 and catheter shaft 107. The interface 150 may be configured to engage the catheter hub 170 and catheter shaft 107 such that rotation of the interface 150 causes rotation of the catheter hub 170 and catheter shaft 107. The clutch mechanism 130 may be actuatable to selectively grip and release the guidewire 105. The guidewire control device 100 is configured such that when the clutch mechanism 130 grips the guidewire 105, the guidewire 105 is prevented from moving rotationally and axially, while the interface 150 and attached catheter hub 170 and catheter shaft 107 freely rotate relative to the housing 110.

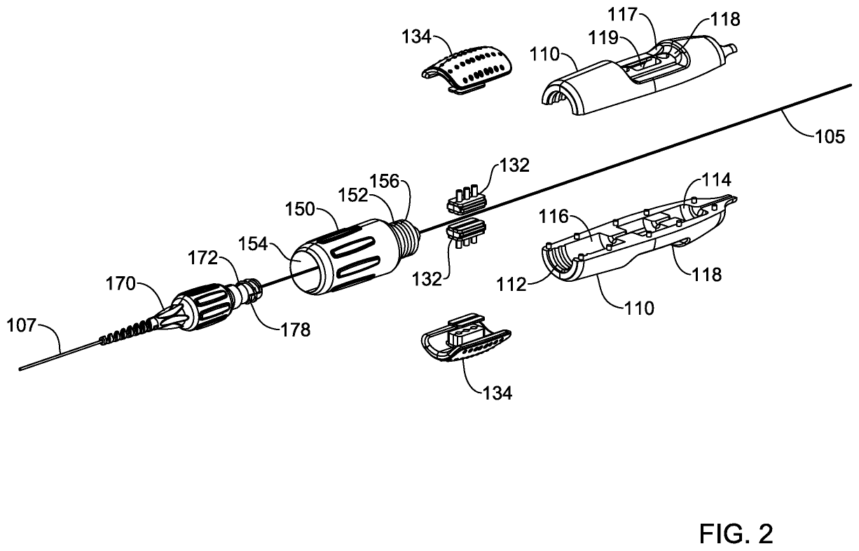

The details of the guidewire control device 100 are illustrated in FIG. 2. The housing 110 may be formed in two parts as shown, or may be formed as a single piece. When the housing 110 is formed in two parts, the parts may be connected by a friction fit, a snap fit, with adhesive, welding, or any other suitable connection. The housing 110 may define a passage 114 extending longitudinally through the housing 110 for receiving the guidewire 105. The passage 114 may be configured to allow the guidewire 105 to freely slide back and forth without obstruction.

The passage 114 may include a chamber 116 at the distal end of the housing 110.

The chamber 116 may be sized and shaped to receive the proximal region 156 of the interface 150 in a rotatable connection such that the interface 150 may freely rotate relative to the housing 110. The inner surface of the chamber 116 may have one or more channel 112 configured to receive one or more protrusion 152 on the outer surface of the proximal region 156 of the interface 150. In other examples, the inner surface of the chamber 116 may have the one or more protrusion and the outer surface of the proximal region 156 of the interface 150 may have the one or more channel. In the illustrated example, the chamber 116 has a plurality of circumferential channels 112 and the protrusions 152 on the proximal region 156 of the interface 150 include a plurality of circumferential ridges 152 configured to rotate within the channels 112. In other examples, the protrusion 152 may be one or more pin, peg, or bump configured to slide within the channel 112. Alternatively, the proximal region 156 of the interface 150 may engage the inner surface of the chamber 116 with a snap fit that allows for free rotation of the interface 150 relative to the housing 110.

The housing 110 may have a pair of recesses 118 in opposing sides. The recesses 118 may be configured to receive the clutch mechanism 130, which may include a pair of clutch grips 134 connected to a pair of engagement members 132. Each recess 118 may have a first portion 117 configured to receive the clutch grip 134, and a second portion 119 extending into the passage 114 and configured to receive the engagement member 132.

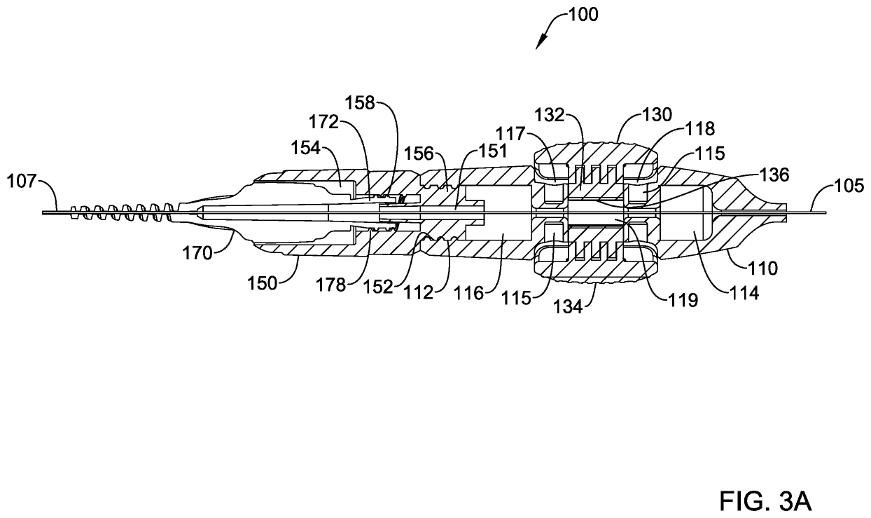

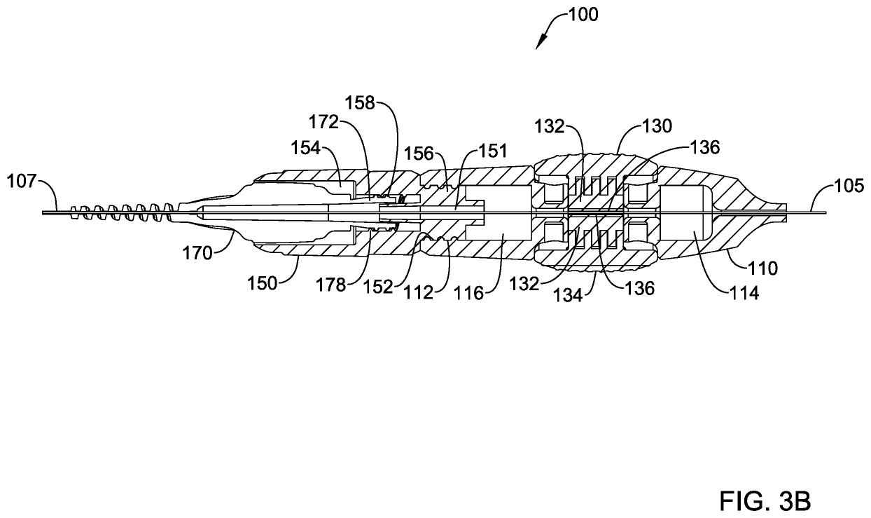

The interface 150 may have a cavity 154 in the distal end configured to receive the proximal end 172 of a catheter hub 170. The catheter hub 170 may be a conventional hub attached to a catheter shaft 107. The proximal end 172 of the catheter hub 170 may have external threading 178 configured to engage internal threading 158 on the inner surface of the cavity 154 of the interface 150, as shown in FIG. 3A. The proximal region 156 of the interface 150 may define a lumen 151 configured for receiving the guidewire 105 such that the guidewire 105 may extend through the housing 110, interface 150, and catheter hub 170. The threading engagement between the interface 150 and catheter hub 170 results in the two parts being rotated together. Rotation of the interface 150 causes equal rotation of the catheter hub 170 and catheter shaft 107. The rotatable connection between the interface 150 and housing 110 allows the interface 150 to be rotated while the housing 110 remains stationary.

The clutch mechanism 130 is actuatable between a release configuration, shown in FIG. 3A, to a gripping configuration, shown in FIG. 3B. In the release configuration shown in FIG. 3A, the clutch grips 134 extend radially outward from the outer surface of the housing 110 and the engagement members 132 are spaced away from the guidewire 105, allowing the guidewire 105 to freely move rotationally and axially within the passage 114 of the housing 110. The clutch grips 134 may be biased in the release configuration. In some examples, a spring element (not shown) may be disposed within the recess 118, holding the clutch grips 134 and the connected engagement members 132 in the release configuration. In one example, a spring element (not shown) may be disposed within cavities 115 in the housing 110 on one or both sides of the engagement members 132, biasing the clutch grips 134 in the release configuration.

Actuating the clutch mechanism 130 may involve pressing down on the clutch grips 134 which moves the engagement members 132 into contact with the guidewire 105, as shown in FIG. 3B. In some examples, the engagement members 132 may be pads with an engagement surface 136 facing the guidewire 105. In some examples, two opposing engagement members 132 may be pressed against one another, capturing the guidewire 105 between engagement surfaces 136 and preventing any rotational or axial movement of the guidewire 105 relative to the housing 110. Actuation of the clutch mechanism 130 may be achieved by gripping the housing 110 in one hand, and squeezing the opposing clutch grips 134 between the fingers and palm. In some examples, the user may squeeze the clutch grips 134 with the ring and/or middle finger and palm, leaving the thumb and first finger free to rotate the interface 150, as will be described below. When the user releases the clutch grips 134, they move back to the release configuration, allowing the guidewire 105 to move freely within the passage 114 of the housing.

In some examples, the engagement surfaces 136 may be textured such that squeezing the engagement surfaces 136 together against the guidewire 105 prevents rotation or axial movement of the guidewire 105, without damaging the guidewire 105. In other examples, the engagement surfaces 136 may be made of a polymer such as silicone. In some examples, the engagement surfaces 136 may include a material that provides an enhanced gripping surface for holding a hydrophilic and/or wet guidewire 105. Examples of such materials include elastomers or tacky materials such as a tacky silicone.

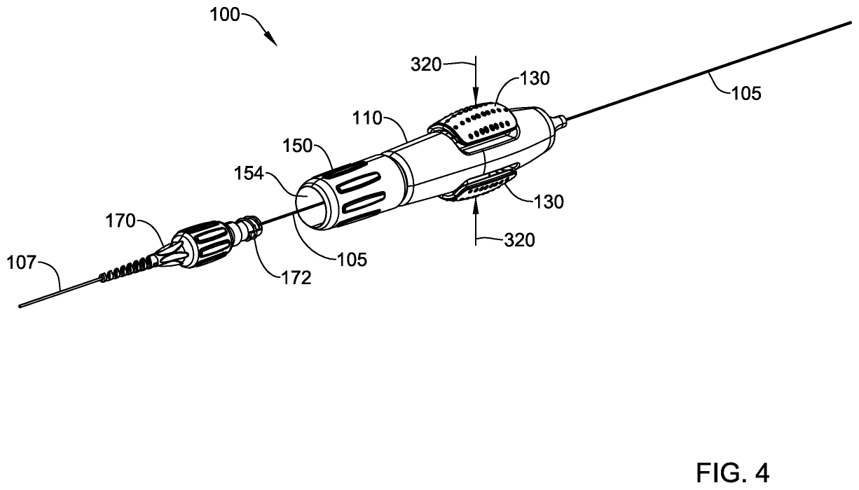

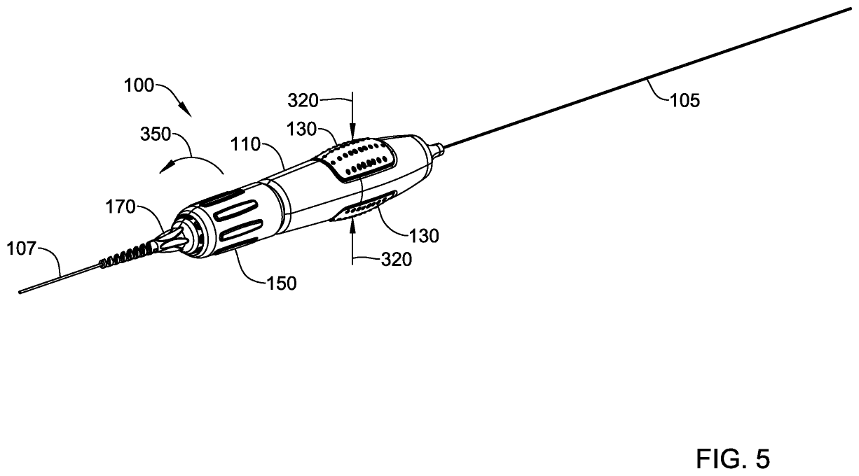

In operation, the user may first load a support catheter shaft 107 attached to a catheter hub 170 over a guidewire 105. Next, the guidewire control device 100, with the clutch mechanism 130 in the release configuration, may be loaded over the guidewire 105 from the proximal end of the guidewire 105, as shown in FIG. 4. The guidewire control device 100 may then be advanced over the catheter hub 170, with the proximal end 172 inserted into the cavity 154 of the interface 150. The interface 150 may then be attached to the catheter hub 170 by threading the interface 150 onto the proximal end 172 of the catheter hub 170 by rotating the interface 150. The clutch mechanism 130 may then actuated to grip the guidewire 105 by applying force in the direction of arrows 320, as shown in FIGS. 4 and 5. With the guidewire control device 100 gripping the guidewire 105, the interface 150 may be rotated as indicated by arrow 350 in FIG. 5, to torque the catheter shaft 107. In some examples, in order to maintain the clutch mechanism 130 in the gripping configuration, shown in FIG. 3B, the force applied in the direction of arrows 320 must be maintained, generally by the user holding the housing 110. When the user releases the clutch mechanism 130, it returns to the release configuration, shown in FIG. 3A. In other examples, pressing the clutch mechanism once may grip the guidewire, and pressing the clutch mechanism a second time may release the guidewire. In still other examples, a release mechanism may be provided to move the clutch mechanism from the gripping configuration to the release configuration.

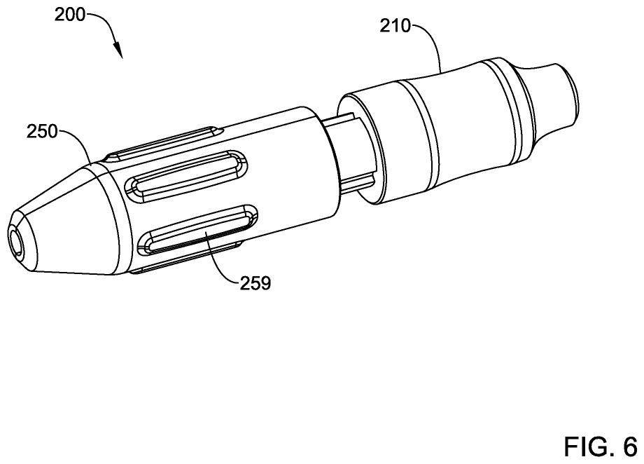

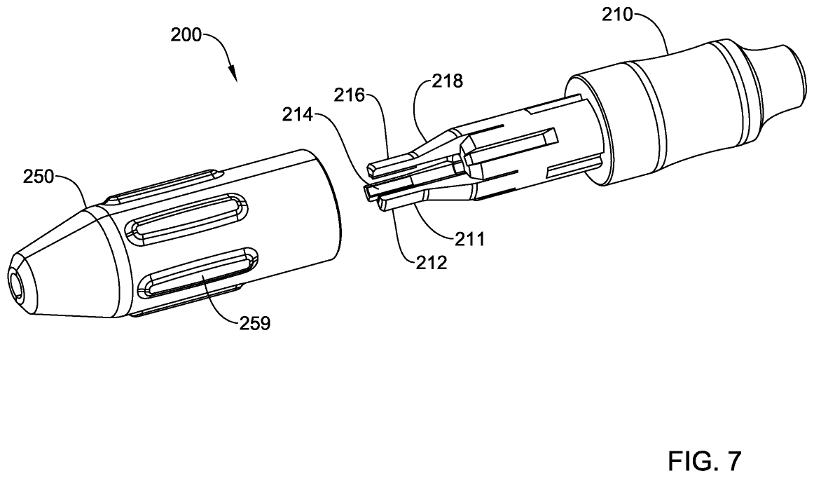

An accessory device 200, as shown in FIGS. 6-9, may be used in combination with the guidewire control device 100 to provide additional torquing of the catheter shaft. The accessory device 200 may include an actuator 210 and a sleeve 250. The sleeve 250 may include one or more protrusions such as ridges 259 shown in FIG. 6. The ridges 259 may provide an enhanced gripping surface to the sleeve 250. As shown in FIG. 7, the distal end of the actuator 210 may include a collet type structure including an actuatable clamp 211 configured to receive a catheter shaft. In some examples, the actuatable clamp 211 may include a plurality of deflectable arms 212 surrounding a lumen 214. Each deflectable arm 212 may include a distal region 216 substantially aligned with a longitudinal axis of the actuator 210, and an angled region 218 proximal of the distal region 216. In the example shown in FIG. 7, the actuator 210 has three deflectable arms 212, however it will be understood that the actuator 210 may include two or more than three deflectable arms 212.

The sleeve 250 may be configured to slide over the actuatable clamp 211 to move the deflectable arms 212 inward toward the lumen 214, into a gripping configuration around the catheter shaft. The plurality of deflectable arms 212 may be moveable between a release configuration in which the catheter slides freely through the lumen 214, and a gripping configuration in which the plurality of deflectable arms 212 engage and grip the catheter shaft and prevent the catheter shaft from moving axially or rotationally relative to the accessory device 200. When in the gripping configuration, rotation of the actuator 210 or the sleeve 250 results in simultaneous and equal rotation of the catheter shaft held within the accessory device 200.

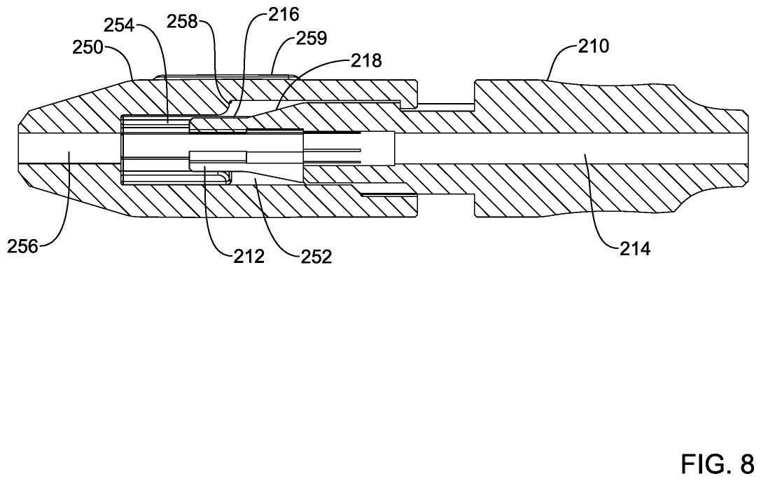

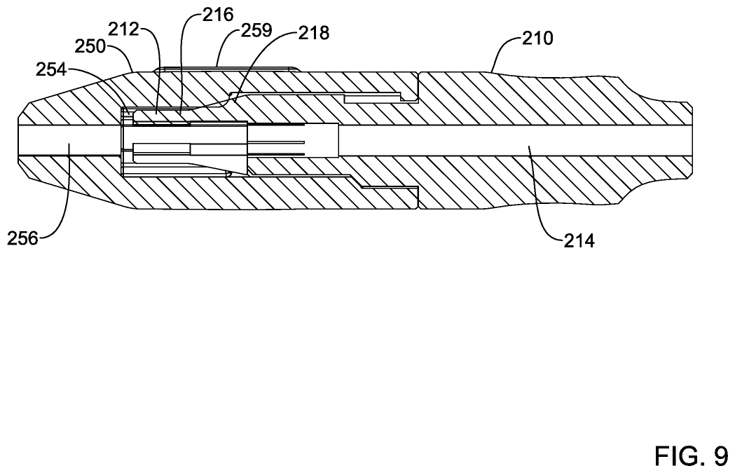

The cross-sectional views in FIGS. 8 and 9 illustrate the details of the actuator 210 and sleeve 250. The sleeve 250 may have a proximal chamber 252 configured to receive the deflectable arms 212 in an expanded or release configuration, and a central chamber 254 configured to receive the deflectable arms 212 in a contracted or gripping configuration. The sleeve 250 may define a lumen 256 extending from a distal end of the sleeve 250 through the central chamber 254 and proximal chamber 252 such that when the sleeve 250 is disposed over the actuator 210, the lumen 256, central chamber 254, proximal chamber 252, and lumen 214 through the actuator 210 define a pathway for receiving a catheter shaft (not shown). In some examples, the inner surfaces of the deflectable arms 212 and/or the lumen 214 of the actuator 210 may have an elastomeric lining (not shown) to aid in gripping the catheter shaft. The proximal chamber 252 may have a first inner diameter and the central chamber 254 may have a second inner diameter. As shown in FIG. 8, the first inner diameter of the proximal chamber 252 may be larger than the second inner diameter of the central chamber 254. A shoulder 258 may be formed at the transition between the central and proximal chambers. The sleeve 250 may be configured to slide over the deflectable arms 212 of the actuator 210. As the distal region 216 of each deflectable arm 212 moves from the proximal chamber 252 into the central chamber 254, the angled region 218 of the deflectable arm 212 engages the shoulder 258, which pushes the angled region 218 of the deflectable arm 212 radially inward, compressing the arms 212 as shown in FIG. 9, clamping a catheter shaft disposed within the lumen 214 of the actuator 210. The catheter shaft is not shown in FIGS. 8 and 9 so as not to obscure the details of the actuator 210 and sleeve 250. However, when present, the catheter shaft would extend through the lumen 256 and central chamber 254 of the sleeve 250, and the lumen 214 of the actuator 210.

The actuator 210 may have the same outer diameter as the sleeve 250, such that when coupled around a catheter shaft, the accessory device 200 provides a uniform outer diameter for gripping and torquing, as shown in FIG. 9. In some examples, one or both of the sleeve 250 and actuator 210 may include one or more protrusions such as the ridges 259 shown on the sleeve 250 in FIG. 6, for aiding the user in gripping and torquing the accessory device 200. In some examples, the accessory device 200 may have an outer diameter the same as or similar to the outer diameter of the catheter hub 170 or interface 150 to enable a user to hold the guidewire control device 100 in one hand and the accessory device 200 in the other hand and apply equal torque on the catheter shaft with both hands when the accessory device 200 is placed on the same catheter shaft as the guidewire control device 100.

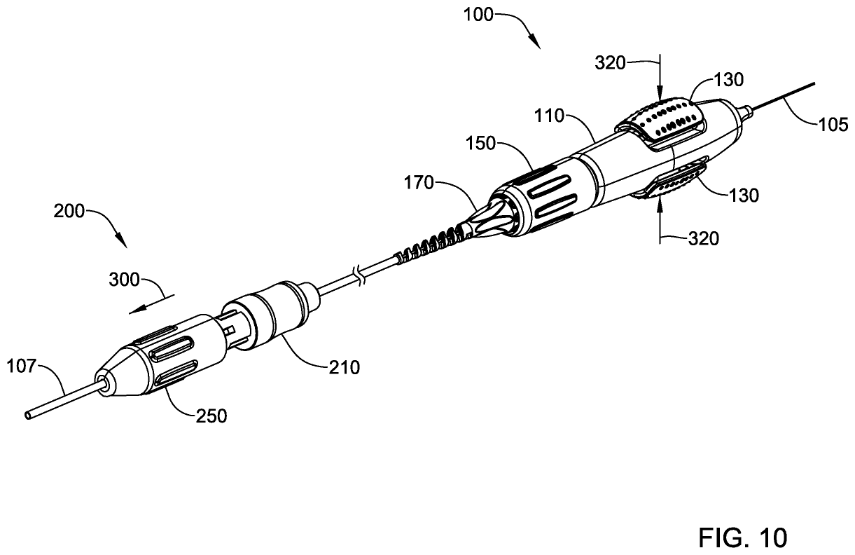

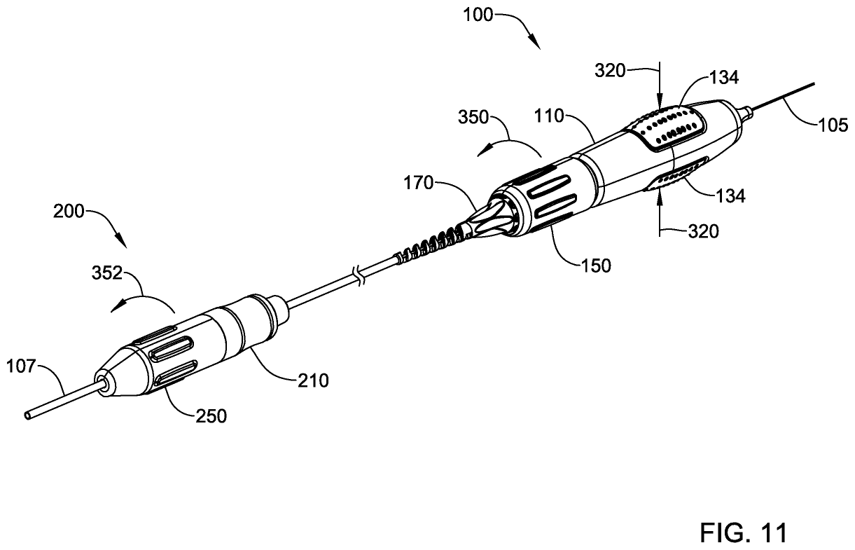

FIGS. 10 and 11 illustrate the guidewire control device 100 and accessory device 200 being attached to the same catheter shaft 107 to allow the user to torque the catheter shaft 107 using both hands while easily holding the guidewire 105 stationary. The accessory device 200 may be loaded onto the catheter shaft 107 while in the release configuration. The accessory device 200 may be loaded onto the catheter shaft 107 with attached catheter hub 170 from the distal end of the catheter shaft 107 before loading the catheter shaft 107 onto the guidewire 105. The accessory device 200 may be actuated to the gripping configuration before or after the guidewire control device 100 is attached. Once the catheter shaft 107, catheter hub 170, and accessory device 200 have been loaded onto the guidewire 105, the guidewire control device 100 may be loaded onto the guidewire 105 from the proximal end of the guidewire 105, and threaded onto the catheter hub 170, as discussed above, with the clutch mechanism 130 in the release configuration. Upon inserting the catheter shaft 107 into the patient, the clutch mechanism 130 may be actuated by applying force in the direction of arrows 320, as discussed above, to grip the guidewire 105 and prevent rotational and axial movement of the guidewire 105 relative to the guidewire control device 100. If the accessory device 200 was not previously actuated to the gripping configuration, the user may then close the gap between the actuator 210 and the sleeve 250 of the accessory device 200 by moving the actuator 210 into the sleeve 250 as indicated by arrow 300. This will actuate the collet design of the deflectable arms 212 to grip the catheter shaft 107 in the gripping configuration.

As shown in FIG. 11, the catheter hub 170 and attached catheter shaft 107 may be torqued by rotating the interface 150 as indicated by arrow 350, and the catheter shaft 107 may be torqued by rotating the accessory device 200 as indicated by arrow 352. The user may grip the housing 110 in the primary hand, holding the clutch grips 134 in the gripping configuration by applying pressure as indicated by arrows 320, to hold the guidewire 105 stationary while the catheter shaft 107 is torqued by rotating the interface 150, such as with the thumb and first finger. The opposite hand may be used to grip and rotate the accessory device 200. The combination of the guidewire control device 100 and accessory device 200 provide the user with improved catheter shaft 107 torquing ability while preventing rotational and axial movement of the guidewire 105. The outer diameters of the interface 150 and accessory device 200 may be the same to minimize differential torque transmission by the two devices. If the guidewire control device 100 needs to be moved relative to the guidewire 105, the user may release the clutch grips 134, allowing the clutch grips 134 to move to the biased, release configuration.

In some examples, the guidewire control device 100 and the accessory device 200 may be made of polymer, elastomeric polyamides, block polyamide/ethers, polyether block amide (PEBA, for example available under the trade name PEBAX®), ethylene tetrafluoroethylene (ETFE), or other polymers generally used in medical catheters.

The materials that can be used for the various components of the guidewire control device 100 and the accessory device 200 (and/or other systems or components disclosed herein) and the various elements thereof disclosed herein may include those commonly associated with medical devices. For simplicity purposes, the following discussion makes reference to the guidewire control device 100 and the accessory device 200 (and variations, systems or components disclosed herein). However, this is not intended to limit the devices and methods described herein, as the discussion may be applied to other elements, members, components, or devices disclosed herein.

In some embodiments, the guidewire control device 100 and the accessory device 200 (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol. In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In some embodiments, the guidewire control device 100 and the accessory device 200 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A guidewire control device comprising:
a housing having a passage for receiving a guidewire;
a clutch connected to the housing and actuatable to selectively grip and release the guidewire disposed in the passage; and
an interface rotatably connected to the housing, the interface configured to receive a catheter hub and torque the catheter hub.

2. The guidewire control device of claim 1, wherein the clutch incudes an engagement member configured to grip the guidewire and prevent rotation of the guidewire while the interface rotates.

3. The guidewire control device of claim 2, wherein the engagement member includes first and second opposing pads configured to be moved toward each other and into contact with the guidewire.

4. The guidewire control device of claim 3, wherein the clutch further includes first and second opposing grips attached to the first and second opposing pads, respectively, the first and second opposing grips configured to move between a release configuration and a gripping configuration, wherein when in the release configuration, the first and second opposing grips extend radially outward from an outer surface of the housing, and when in the gripping configuration, the first and second opposing grips are disposed radially inward toward the passage.

5. The guidewire control device of claim 4, wherein the first and second opposing grips are biased in the release configuration.

6. The guidewire control device of claim 1, wherein the interface includes a proximal end defining a lumen for receiving the guidewire, the proximal end having at least one protrusion configured to rotate within at least one channel on an inner surface of the passage of the housing.

7. The guidewire control device of claim 6, wherein the interface includes a distal end defining a cavity having internal threading configured to receive an externally threaded proximal end of the catheter hub.

8. The guidewire control device of claim 1, further comprising an accessory device including an actuator having a lumen configured to receive a catheter, the actuator having a distal end defining an actuatable clamp configured to engage the catheter, the accessory device further including a sleeve configured to actuate the actuatable clamp.

9. The guidewire control device of claim 8, wherein the actuatable clamp includes a plurality of deflectable arms moveable between a release configuration in which the catheter slides through the lumen, and a gripping configuration in which the plurality of deflectable arms engage the catheter and prevent the catheter from moving axially or rotationally.

10. The guidewire control device of claim 9, wherein the sleeve defines a proximal chamber configured to receive the plurality of deflectable arms in the release configuration, a central chamber configured to receive the plurality of deflectable arms in the gripping configuration, and a lumen configured to receive the catheter, wherein when the plurality of deflectable arms of the actuator are moved from the proximal chamber into the central chamber, the plurality of deflectable arms are compressed radially inward into engagement with the catheter.

11. A guidewire control assembly comprising:
a guidewire clamping device including:
a proximal housing having a passage for receiving a guidewire;
a clutch connected to the proximal housing and actuatable to selectively grip and release the guidewire disposed in the passage; and
a distal housing rotatably connected to the proximal housing, the distal housing configured to receive a catheter hub;
a catheter clamping device including a first member having a lumen configured to receive a catheter and a distal end defining an actuatable clamp configured to engage the catheter, the catheter clamping device further including a second member having a cavity configured to receive and actuate the actuatable clamp.

12. The guidewire control assembly of claim 11, wherein the clutch incudes an engagement member configured to grip the guidewire and prevent rotation of the guidewire while the distal housing rotates, the engagement member including first and second opposing pads configured to be moved toward each other and into contact with the guidewire.

13. The guidewire control assembly of claim 12, wherein the clutch further includes first and second opposing grips attached to the first and second opposing pads, respectively, the first and second opposing grips configured to move between a release configuration and a gripping configuration, wherein when in the release configuration, the first and second opposing grips extend radially outward from an outer surface of the proximal housing, and when in the gripping configuration, the first and second opposing grips are disposed radially inward toward the passage.

14. The guidewire control assembly of claim 13, wherein the first and second opposing grips are biased in the release configuration.

15. The guidewire control assembly of claim 11, wherein the distal housing includes a proximal end defining a lumen for receiving the guidewire, the proximal end having at least one protrusion configured to rotate within at least one channel on an inner surface of the passage of the distal housing.

16. The guidewire control assembly of claim 15, wherein the distal housing includes a distal end defining a cavity having internal threading configured to receive an externally threaded proximal end of the catheter hub.

17. The guidewire control assembly of claim 11, wherein the actuatable clamp includes a plurality of deflectable arms moveable between a release configuration in which the catheter slides through the lumen, and a gripping configuration in which the plurality of deflectable arms engage the catheter and prevent the catheter from moving axially or rotationally.

18. The guidewire control assembly of claim 17, wherein the second member defines a lumen having a proximal region configured to receive the plurality of deflectable arms in the release configuration a middle region configured to receive the plurality of deflectable arms in the gripping configuration, and a distal region configured to receive the catheter, wherein when the plurality of deflectable arms of the actuatable clamp are moved from the proximal region into the middle region, the plurality of deflectable arms are compressed radially inward into engagement with the catheter.

19. A method of torquing a catheter disposed over a guidewire, the method comprising:
loading a catheter shaft attached to a catheter hub over a guidewire;
loading a guidewire control device onto the guidewire, the guidewire control device including a housing having a passage for receiving the guidewire, a clutch connected to the housing and actuatable between a release configuration in which the guidewire is freely moveable within the passage and a gripping configuration in which the guidewire is prevented from rotational and axial movement relative to the housing, and an interface rotatably connected to the housing, wherein the clutch is in the release configuration;
attaching the catheter hub to the interface;
actuating the clutch to the gripping configuration thereby preventing rotational and axial movement of the guidewire relative to the housing; and
torquing the catheter by rotating the interface.

20. The method of claim 19, wherein before loading the catheter shaft over the guidewire, the method further comprises:
loading an accessory device onto the catheter shaft, the accessory device including an actuator having a lumen configured to receive the catheter shaft, the actuator having a distal end defining an actuatable clamp having a release configuration in which the catheter shaft is freely moveable within the lumen and a gripping configuration in which the catheter shaft is prevented from rotational and axial movement relative to the accessory device, the accessory device further including a sleeve configured to actuate the actuatable clamp, wherein the actuatable clamp is in the release configuration;
actuating the accessory device by moving the sleeve over the actuatable clamp thereby moving the actuatable clamp into the gripping configuration; and
wherein torquing the catheter includes rotating the interface and the accessory device in the same direction.
